(19) United States
(12) Patent Application Publication
(10) Pub. No.: US 2002/0029429 A1
(43) Pub. Date: Mar. 14, 2002

(54) HAIR COLORING COMPOSITIONS

(76) Inventors: LOUIS CARLOS DIAS, WEYBRIDGE (GB); JAMES CHARLES DUNBAR, WEYBRIDGE (GB); DOMINIC PRATT, KINGSON UPON THAMES (GB)

Correspondence Address: S T MURPHY, THE PROCTER & GAMBLE CO PATENT DIVISION, SHARON WOODS TECHNICAL CENTER, 11510 REED HARTMAN HIGHWAY, CINCINNATI, OH, 45241

(21) Appl. No.: 09/331,681

(22) PCT Filed: Dec. 9, 1997
(86) PCT No.: PCT/US97/22715

(30) Foreign Application Priority Data
Dec. 23, 1996 (GB) 9626712.5

(51) Int. Cl. A61K 7/13

(52) U.S. Cl. 8/405; 8/407; 8/411; 8/421

(57) ABSTRACT

A storage stable, re-usable hair coloring composition comprising: (a) an oxidizing agent; and (b) an oxidative hair coloring agent; wherein the pH of each of (a) and (b) is in the range of from about 1 to about 6 and wherein the combined mixture of (a) and (b) has a pH in the range of from about 1 to about 5 and wherein both (a) and (b) are capable of being stored at low pH, either separately, or, once mixed, for at least 1 month at room temperature and wherein the resultant color delivered to the hair (Delta E) is up to about 75%, preferably up to about 85%, more preferably up to about 90% and most preferably up to about 95% of the total color delivered to the hair (Delta E) from a mixture of (a) and (b) on mixing. The products are storage stable and re-usable and can provide excellent hair coloring and in-use efficacy benefits in combination with improved color retention potential after storage at room temperature.

### TECHNICAL FIELD

This invention relates to hair coloring compositions and processes for coloring hair, and more especially to hair coloring compositions comprising either, (i) a separate, storage stable, oxidising agent component in combination with a separate, storage stable, oxidative hair coloring component, each at an acidic pH of between about 1 and about 6, which, when combined, result in a combined, storage stable, re-usable, coloring composition having a pH of between about 1 and about 5, or, (ii) a storage stable, re-usable mixture of oxidising agent and oxidative hair coloring agent at a combined acidic pH of between about 1 and about 5.

### BACKGROUND OF THE INVENTION

The desire to alter the color of human hair is not a facet of modern times. Since the days of the Roman Empire the color of human hair has been routinely altered to accommodate the changes of fashion and style. However the attainment of precise initial colors which are retained by the hair for a desirable period has remained a more elusive goal. The difficulties in the development of hair coloring compositions which can deliver precise long-lasting colors are in part due to the inherent structure of the hair itself and in part due to the necessary conditions of effective hair coloration processes.

Over the years significant effort has been directed towards the elimination of many of the problems associated with the dyeing of human hair. Various approaches to hair dyeing have been developed, these include, direct action dyes, natural dyes, metallic dyes and oxidative dyes.

To color human or animal hair using oxidative dye technology the hair is generally treated with a mixture of oxidative hair coloring agents and an oxidising agent.

Oxidative hair coloring agents and oxidising agents can be used to deliver a variety of hair colors to the hair. However substantial improvement is needed in the area of application characteristics of coloring compositions, such as, time to prepare the coloring composition, mess on application, waste of product not used, waste of packaging (from use of multiple component systems), storage stability (of the coloring composition once mixed), and the ability to re-use the mixed coloring composition. Re-use, of a coloring composition, as defined herein, means, correction of mistakes, touching up or root coverage, and future re-coloring of the hair with the same product as well as color and/or strand tests on small areas of hair.

In addition to the improvements in application charateristics as detailed hereinbefore, substantial improvement is needed in the area of coloring characteristics such as, color saturation, color development, precise initial color consistency, improved wash fastness, improved hair condition and levels of hair damage.

Conventional oxidative hair dyeing compositions generally comprise at least two separately packaged components. Typically, these components include oxidising agent (at low pH), such as hydrogen peroxide, and dyeing material (at high pH), such as oxidative hair coloring agents. In order to facilitate the hair dyeing process these separately packaged components are generally admixed just prior to application of the coloring composition to the hair. After mixing, such, high pH, compositions tend to degrade which can lead to reduced or impaired coloring ability. High pH hair coloring compositions are typically used soon after admixing. In general, any excess, admixed, coloring composition is disposed of after application of the required amount to the hair. Furthermore, such conventional coloring compositions, once mixed, cannot be stored and/or re-used, as the activated oxidative dyeing system rapidly deteriorates over time. Thus, it would be desirable to develop an oxidative hair coloring composition which comprises both oxidising agent and oxidative coloring agents which is storage stable and/or re-useable.

As detailed hereinbefore conventional oxidative hair dyeing compositions generally comprise at least two separately packaged components. In addition to the time required to effectively mix these components, the mixing process itself can be both messy and wasteful as well as causing added inconvenience to the user. Thus, it would be desirable to develop a convenient and easy-to-use method for the delivery of oxidative hair coloring compositions to the hair which does not require mixing of the oxidising and coloring components.

It would also be desirable to develop a hair coloring composition comprising an oxidising agent and an oxidative hair coloring agent which can be stored as separately packaged, stable, low pH, components which, on mixing, provides a shelf-stable, low pH mixture which is suitable for direct application to the hair. This low pH mixture could be stored and re-used. This would allow the consumer to correct mistakes, touch up roots or re-apply product without re-purchase.

It has been found that, at low pH, mixtures of oxidising agents and oxidative hair coloring agents, can be packaged together, in a single pack, and are storage stable and re-usable. It has also been found that such low pH mixtures of oxidising agent and oxidative hair coloring agents provide improved initial hair coloring attributes versus conventional, high pH, compositions. It has also been found that, low pH mixtures of oxidising agent and oxidative hair coloring agents, which have been stored at room temperature, provide improved color development potential versus conventional coloring compositions.

It has also been found that, at low pH, both the oxidising agent and oxidative hair coloring agents are stable over time, and can be stored as such.

It is an object of the present invention to provide hair coloring compositions comprising stable, separately packaged oxidant and oxidative hair coloring agent which remain stable at low pH both, when stored individually, or, when mixed, and which have excellent initial hair coloring characteristics and/or color retention potential.

It is a further object of the present invention to provide, low pH hair coloring compositions comprising both oxidising agents and oxidative hair coloring agent which are singly packaged and/or re-usable, and which are fast acting, simple to use and storage stable.

It has been found that the above objects can be met by the low pH, storage stable, re-usable hair coloring compositions according to the present invention.

All percentages are by weight of the final compositions in the form intended to be used unless specified otherwise.

### SUMMARY OF THE INVENTION

The subject of the present invention is a hair coloring composition suitable for the treatment of human or animal hair.

According to one aspect of the present invention, there is provided a storage stable, re-usable hair coloring composition comprising:

(a) an oxidising agent; and

(b) an oxidative hair coloring agent;

wherein the pH of each of (a) and (b) is in the range of from about 1 to about 6 and wherein the combined mixture of (a) and (b) has a pH in the range of from about 1 to about 5 and wherein both (a) and (b) are capable of being stored at low pH, either separately, or, once mixed, for at least 1 month at room temperature and wherein the resultant color delivered to the hair (Delta E) is up to about 75%, preferably up to about 85%, more preferably up to about 90% and most preferably up to about 95% of the total color delivered to the hair (Delta E) from a mixture of (a) and (b) on mixing.

It is to be understood that the percentage weights of the composition components herein are expressed in terms of the total composition, and includes, the composition when in the form of intended use.

According to a further aspect of the present invention, there is provided:

A method for coloring hair wherein a hair coloring mixture is present in a single package suitable for direct application to the hair wherein the hair coloring mixture comprises:

(a) an oxidising agent; and

(b) an oxidative hair coloring agent;

wherein the combined mixture of (a) and (b) has a pH in the range of from about 1 to about 5 and wherein the resultant color delivered to the hair (Delta E), after at least about 1 month of storage at room temperature, is up to about 75%, preferably up to about 85%, more preferably up to about 90% and most preferably up to about 95% of the total color delivered to the hair (Delta E) from a mixture of (a) and (b) on mixing.

According to an additional object of the present invention there is provided a hair coloring composition comprising:

(a) an oxidising agent; and

(b) an oxidative hair coloring agent;

wherein the pH of each of (a) and (b) is in the range of from about 1 to about 6, wherein the combined mixture of (a) and (b) has a pH in the range of from about 1 to about 5, wherein (a) and (b) are in the form of intended use, and wherein the molar level of (a) is from about from about 0.0003 moles (per 100 g of composition) to less than about 0.2 moles (per 100 g of composition) and wherein both (a) and (b) are capable of being stored at low pH, either separately or once mixed and wherein the resultant color delivered to the hair (Delta E), after at least about 1 month of storage, is up to about 75%, preferably up to about 85%, more preferably up to about 90% and most preferably up to about 95% of the total color delivered to the hair (Delta E) from a mixture of (a) and (b) on mixing.

According to a yet further aspect of the present invention there is provided a method for coloring hair wherein a hair coloring composition is present as separately packaged components (a) and (b) and wherein the hair coloring composition comprises:

(a) from about 0.0003 moles (per 100 g of composition) to less than about 0.2 moles (per 100 g of composition) of an oxidising agent wherein the pH of (a), is in the range of from about 1 to about 6; and

(b) an oxidative hair coloring agent wherein the pH of (b), is in the range of from about 1 to about 6;

wherein each of (a) and (b), when in the form of intended use, are stable at pHs in the range of from about 1 to about 6 and wherein the combined hair coloring mixture of (a) and (b) is stable over time and has a pH in the range of from about 1 to about 5 and wherein the resultant color delivered to the hair (Delta E), after at least about 1 month of storage at room temperature, is up to about 75%, preferably up to about 85%, more preferably up to about 90% and most preferably up to about 95% of the total color delivered to the hair (Delta E) from a mixture of (a) and (b) after 1 hour of storage.

### DETAILED DESCRIPTION OF THE INVENTION

As used herein the term ‘hair’ to be treated may be ‘living’ i.e. on a living body or may be ‘non-living’ i.e. in a wig, hairpiece or other aggregation of non-living fibres, such as those used in textiles and fabrics. Mammalian, preferably human hair is preferred. However wool, fur and other melanin containing fibres are suitable substrates for the compositions according to the present invention.

As used herein the term ‘hair coloring composition’ is used in the broad sense in that it is intended to encompass compositions containing the combinations herein of a low pH (from about 1 to about 5) mixture of oxidising agent and an oxidative coloring agent. Moreover, it is also intended to include complex compositions which contain other components which may or may not be active ingredients. Thus, the term ‘hair coloring composition’ is intended to apply to compositions which contain, in addition to a mixture of active oxidising agents and oxidative coloring agents, such things as, by way of example, oxidising aids, sequestrants, stabilisers, thickeners, buffers, carriers, surfactants, solvents, antioxidants, polymers, non-oxidative dyes and conditioners.

As discussed above, the low pH, storage stable and/or re-usable hair coloring compositions according to the present invention comprise an oxidising agent (a), which is packaged either separately, or, in combination with an oxidative hair coloring agent (b) wherein each of (a) and (b), when in the form of intended use, is storage stable at pHs in the range of from about pH 1 to about pH 6 and wherein the combined mixture of (a) and (b) is storage stable in the pH range of from about 1 to about 6. Preferably, the pH of either (a) and/or (b) is in the range of from about about 1.5 to about 5.8, more preferably from about 1.8 to about 5.5, most preferably from about 2 to about 5 and especially from about 3.5 to about 4.5 and wherein the preferred pH of the combined mixture of (a) and (b) is in the range of from about 1.5 to about 5, more preferably from about 1.8 to about 4.7, most preferably from about 2 to about 4.6, especially from about 2.5 to about 4.5 and most especially from 2.7 to 3.8.

### The Dye Oxidisation and Hair Coloring Processes

It is understood by those familiar in the art that to successfully color human or animal hair with oxidative dyes it is generally necessary to treat the hair with a mixture of oxidising agent and oxidative hair coloring agent. As herein before discussed the most common oxidising agent is hydrogen peroxide.

Hydrogen peroxide has a pKa in the range of from about 11.2 to about 11.6, and, as such is generally used as a dye oxidising agent at pHs in the range of from about 9 to about 12.

Surprisingly, it has now been found that, at low pH, storage stable and/or re-usable oxidative hair coloring compositions can be developed. Furthermore, it has also been found that improved hair coloring characteristics, such as, initial color development, and improved color washfastness, are provided by the low pH, storage stable and/or re-usable oxidative hair coloring compositions of the present invention, at pHs in the range of from about pH 1 to about pH 5, preferably from about pH 1.5 to about 5, more preferably from about 1.8 to about 4.7, most preferably from about 2 to about 4.6, especially from about 2.5 to about 4.5 and most especially from 2.7 to 3.8.

### Color Stable Low pH Storage Stable Compositions

The compositions according to the present invention comprise as essential features an oxidising agent and an oxidative hair coloring agent at low pH wherein both the oxidisng agent component and the oxidative hair coloring component are present, when in the form of intended use, at a pH in the range of from about pH to about pH 6 and wherein the combined mixture of oxidising agent and oxidative hair coloring agent has a pH in the range of from about pH 1 to about pH 5.

As hereinbefore described the mixture of the individual oxidising and hair coloring components of the hair coloring compositions according to the present invention have improved storage stability under low pH conditions (about pH 1 to about pH 5) versus conventional, high pH, coloring compositions. In addition, it has been found that oxidative hair coloring agents can be separately stored, at low pH, and admixed with oxidising agent, at low pH, to form a storage stable, re-usable, hair coloring composition.

Storage stable hair coloring composition, as defined herein, includes, a coloring composition comprising an oxidising component and an oxidative hair coloring component, which, once mixed, retain the ability to develop a consistent/predictable initial hair color both imediately after mixing, and, after storage over time. Also defined as storage stable hair coloring compositions herein are single component coloring compositions which are suitable fo direct application to the hair which comprise a pre-mixed combination of an oxidising agent and an oxidative hair coloring agent.

In terms of consumer perception, consistent/predictable initial color, means, that the color delivered to the hair, by the stored composition (Delta E stored), is not visibly different from the color delivered by the composition on formulation (first mixing of the oxidising agent and oxidative coloring agent) and/or at point of purchase (when sold as a single component mixture). Both initial hair coloring and hair coloring after storage, as will be explained in detail hereinafter, can be measured in terms of Delta E (Delta E target, Delta E stored). Retained color development potential, as defined herein, means, that the color developed by the hair coloring composition on first mixing (Delta E target) is substantially delivered to the hair after extended storage of the composition over time.

In compositions according to the present invention the color delivered to the hair after at least about 1 hour (60 minutes) of storage (Delta E stored) at room temperature (25° C.) is greater than about 75%, preferably greater than about 80%, more preferably greater than about 85%, most preferably greater than about 90% and especially greater than about 95% of the color delivered to the hair on mixing (Delta E target).

In preferred compositions according to the present invention the color delivered to the hair at least about 1 day (24 hours) of storage (Delta E stored) at room temperature (25° C.) is greater than about 75%, preferably greater than about 80%, more preferably greater than about 85%, most preferably greater than about 90% and especially greater than about 95% of the color delivered to the hair on mixing (Delta E target).

In highly preferred compositions according to the present invention the color delivered to the hair after at least about 1 month (720 hours) of storage (Delta E stored) at room temperature (25° C.) is greater than about 75%, preferably greater than about 80%, more preferably greater than about 85%, most preferably greater than about 90% and especially greater than about 95% of the color delivered to the hair on mixing (Delta E target).

According to one aspect of the present invention, there is provided a storage stable, re-usable hair coloring composition comprising:

(a) an oxidising agent; and

(b) an oxidative hair coloring agent;

wherein the pH of each of (a) and (b) is in the range of from about 1 to about 6 and wherein the combined mixture of (a) and (b) has a pH in the range of from about 1 to about 5 and wherein both (a) and (b) are capable of being stored at low pH, either separately or once mixed at least 1 about month and wherein the resultant color delivered to the hair (Delta E storage) is greater than about 75%, preferably greater than about 85%, more preferably greater than about 90% and most preferably greater than about 95% of the total color delivered to the hair (Delta E target) from a mixture of (a) and (b) on mixing.

It is generally accepted that the pH within the hair shaft, of human hair, is around pH 5.5 to pH 6 (C. R. Robbins, Chemical and Physical Behaviour of Human Hair, 2nd Ed. p157) and that human hair has an inherent buffering capacity. It is known that certain oxidising agents, such as hydrogen peroxide, are storage stable at about pH 4. While not wishing to be bound to any particular theory, it is believed that at pHs in the range of from about 1 to about 5, preferably in the range of from about 1.5 to about 5, more preferably from about 1.8 to about 4.7, most preferably from about 2 to about 4.6, especially from about 2.5 to about 4.5 and most especially from 2.7 to 3.8, the rate of oxidative coupling, between the oxidising agent and the oxidative hair coloring agents is relatively slow. As such, it is proposed herein that, at low pH, mixtures of oxidative hair coloring agents and oxidising agents can be stored stably without significant impact on the color development potential of the resultant hair coloring composition.

The singly packaged, re-usable, low pH oxidative hair coloring compositions of the present invention are also suitable for use in a multi-application format (i.e. the consumer can use a single package for several color applications over a period of time) which give the consumer the ability to re-use the mixed coloring composition for, correction of mistakes, touching up or root coverage, future re-coloring of the hair with the same product/color and/or strand tests on small areas of hair. Singly packaged, as defined herein, means, a hair coloring composition comprising a single packaging component which contains therein oxidising agent and oxidative coloring agent together in a stable, low pH mixture.

### Component Parts of Hair Coloring Compositions

As detailed hereinbefore it has been found that oxidative hair coloring materials can be stored at low pH with excellent retained color development potential. As defined herein, retained coloring potential for the oxidative coloring component (b), means, that the color as developed, on mixing the oxidative hair coloring component (b) with the oxidising agent (a), at time zero (i.e. Delta E target), is approximately equivalent to the color as developed from the mixture of the oxidative hair coloring component (b) with the oxidising agent (a), after a period of storage (Delta E stored) at room temperature, 25° C., for X hours.

In preferred compositions according to the present invention Delta E (X hours) is greater than about 90%, preferably greater than about 93%, more preferably greater than about 95% and most preferably greater than about 98% of Delta E (target).

Thus according to another aspect of the present invention there is provided a storage stable, re-usable hair coloring composition comprising:

(a) an oxidising agent; and

(b) an oxidative hair coloring agent;

wherein the pH of each of (a) and (b) is in the range of from about 1 to about 6 and wherein the combined mixture of (a) and (b) has a pH in the range of from about 1 to about 5 and wherein both (a) and (b) are capable of being stored at low pH, at room temperature (about 25° C.), either separately or once mixed, for at least about 1 month and wherein the color potential delivered to the hair (Delta E) is greater than about 75%, preferably greater than about 85%, more preferably greater than about 90% and most preferably greater than about 95% of the total color delivered to the hair (Delta E) from a mixture of (a) and (b) on mixing.

### Oxidising Agents

The compositions of the invention comprise as an essential feature at least one oxidising agent. The oxidising agent should be safe and effective for use in the compositions herein. Preferably, the oxidising agents suitable for use herein will be soluble in the compositions according to the present invention when in liquid form and/or in the form intended to be used. Preferably, oxidising agents suitable for use herein will be water-soluble. Water soluble oxidising agents as defined herein means agents which have a solubility to the extent of about 10 g in 1000 ml of deionised water at 25° C. (“Chemistry” C. E. Mortimer. 5th Edn. p277).

Suitable oxidising agents for use herein are selected from inorganic peroxygen oxidising agents, preformed organic peroxyacid oxidising agents and organic peroxide oxidising agents or mixtures thereof.

### Inorganic Oxidising Agents

The compositions of the invention may comprise at least one inorganic oxidising agent (hereinafter called ‘inorganic peroxygen oxidising agent’). The inorganic peroxygen oxidising agent should be safe and effective for use in the compositions herein. Preferably, the inorganic peroxygen oxidising agents suitable for use herein will be soluble in the compositions according to the present invention when in liquid form and/or in the form intended to be used. Preferably, inorganic peroxygen oxidising agents suitable for use herein will be water-soluble. Water soluble inorganic peroxygen oxidising agents as defined herein means agents which have a solubility to the extent of about 10 g in 1000 ml of deionised water at 25° C. (“Chemistry” C. E. Mortimer. 5th Edn. p277).

The inorganic peroxygen oxidising agents useful herein are generally inorganic peroxygen materials capable of yielding peroxide in an aqueous solution. Inorganic peroxygen oxidising agents are well known in the art and include hydrogen peroxide, inorganic alkali metal peroxides such as sodium periodate, sodium perbromate and sodium peroxide, and inorganic perhydrate salt oxidising compounds, such as the alkali metal salts of perborates, percarbonates, perphosphates, persilicates, persulphates and the like. These inorganic perhydrate salts may be incorporated as monohydrates, tetrahydrates etc. Mixtures of two or more of such inorganic peroxygen oxidising agents can be used if desired. While alkali metal bromates and iodates are suitable for use herein the bromates are preferred. Highly preferred for use in the compositions according to the present invention is hydrogen peroxide.

It has also been found that, under the low pH conditions according to the present invention, it is possible to deliver both improved initial color development, color consistency, washfastness and color intensity versus conventional, high pH, systems (using equivalent levels of peroxide and dyes) and also equivalent color development (detailed hereinafter in the Experimental Data section in terms of Delta E) versus conventional systems at high pH, while using substantially less oxidising agent (up to 75% less) as well as delivering equivalent color development versus conventional, high pH, systems while using substantially less oxidative hair coloring agent (up to 50% less). Thus, the storage stable and/or re-usable, low pH, hair coloring compositions according to the present invention can be formulated to reduce the level of damage to the hair and levels of skin irritation and staining. Furthermore, as the storage stable and/or re-usable, low pH, hair coloring compositions according to the present invention can be formulated without ammonia there are no ammonia related odor or skin irritation negatives associated with these compositions.

The inorganic peroxygen oxidising agent where present, is present in the compositions according to the present invention at a molar level of from about 0.0003 moles (per 100 g of composition) to less than about 0.2 moles (per 100 g of composition), preferably, the inorganic peroxygen oxidising agent is present at a molar level of from about 0.006 moles to about 0.15 moles, more preferably from about 0.009 moles to about 0.12 moles, most preferably from about 0.012 moles to about 0.1 moles, from about 0.015 mole to about 0.09 moles (per 100 g of composition).

The inorganic peroxygen oxidising agent, where present, is present in the compositions of the present invention at a level of from about 0.01% to about 6%, preferably from about 0.1% to about 5%, more preferably from about 0.2% to about 4%, most preferably from about 0.3% to about 3% and especially from about 0.5% to about 2% by weight.

### Preformed Organic Peroxyacid

The compositions according to the present invention may contain one or more preformed organic peroxyacid oxidising agents.

Suitable organic peroxyacid oxidising agents for use in the coloring compositions according to the present invention have the general formula:

R—C(O)OOH

wherein R is selected from saturated or unsaturated, substituted or unsubstituted, straight or branched chain, alkyl, aryl or alkaryl groups with from 1 to 14 carbon atoms.

A class of organic peroxyacid compounds suitable for use herein are the amide substituted compounds of the following general formulae:

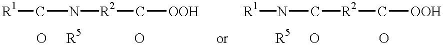

wherein R¹ is, a saturated or unsaturated alkyl or alkaryl group, or an aryl group, having from 1 to 14 carbon atoms, R² is, a saturated or unsaturated alkyl or alkaryl group, or an aryl group, having from 1 to 14 carbon atoms, and R⁵ is H or, a saturated or unsaturated alkyl or alkaryl group, or an aryl group, having from 1 to 10 carbon atoms. Amide substituted organic peroxyacid compounds of this type are described in EP-A-0,170,386.

Other suitable organic peroxyacid oxidising agents include peracetic, pernanoic, nonylamidoperoxycaproic acid (NAPCA), perbenzoic, m-chloroperbenzoic, di-peroxy-isophthalic, mono-peroxyphthalic, peroxylauric, hexanesulphonyl peroxy propionic, N,N-phthaloylamino peroxycaproic, monoper succinic, nonanoyloxybenzoic, dodecanedioylmonoperoxybenzoic, nonylamide of peroxyadipic acid, diacyl and tetraacylperoxides, especially diperoxydodecanedioic acid, diperoxytetradecanedioic acid and diperoxyhexadecanedioic acid and derivatives thereof. Mono- and diperazelaic acid, mono- and diperbrassylic acid and N-phthaloylaminoperoxicaproic acid and derivatives thereof are also suitable for use herein.

The preferred peroxyacid materials suitable for use herein are selected from peracetic and pernanoic acids and mixtures thereof.

The preformed organic peroxyacid oxidising agents should be safe and effective for use in the compositions herein. Preferably, the preformed organic peroxyacid oxidising agents suitable for use herein will be soluble in the compositions according to the present invention when in liquid form and/or in the form intended to be used. Preferably, preformed organic peroxyacid oxidising agents suitable for use herein will be water-soluble. Water soluble preformed organic peroxyacid oxidising agents as defined herein means agents which have a solubility to the extent of about 10 g in 1000 ml of deionised water at 25° C. (“Chemistry” C. E. Mortimer. 5th Edn. p277).

The preformed organic peroxyacid oxidising agent, where present, is present at a molar level of from about 0.00013 moles to about 0.105 moles (per 100 g) of composition), more preferably from about 0.0013 moles to about 0.05 moles, most preferably from about 0.002 moles to about 0.04 moles and especially from about 0.004 moles to about 0.03 moles (per 100 g) of the hair coloring compositions according to the present invention.

The preformed organic peroxyacid oxidising agent is preferably present at a level of from about 0.01% to about 8%, more preferably from about 0.1% to about 6%, most preferably from about 0.2% to about 4%, and especially from about 0.3% to about 3% by weight of the hair coloring composition. The weight ratio of the inorganic peroxygen oxidising agent to the preformed organic peroxy acid is preferably in the range of from about 0.0125:1 to about 500:1, more preferably from about 0.0125:1 to about 50:1.

In addition to preformed organic peroxyacid oxidising agents and the inorganic peroxygen oxidising agents suitable for use herein, the compositions according to the present invention, may, comprise additional organic peroxides such as urea peroxide, melamine peroxide and mixtures thereof. The level of organic peroxide, where present, is from about 0.01% to about 3%, preferably from about 0.01% to about 2%, more preferably from about 0.1% to about 1.5% and most preferably from about 0.2% to about 1% by weight of composition.

### Hair Coloring

Once hair has been colored there is a desire for the color to be resistant to fading, as occasioned by the actions of washing (also known as wash fastness), perspiration, hair spray and other exterior factors such as the action of the sun, and further that the color be retained in a consistent manner for a predictable period of time. Additionally damage to the hair that can lead to irregular dye uptake as discussed above, can lead to increased fading of the damaged portions of the hair and consequently, irregular levels of color fade over time. An additional difficulty commonly associated with the dyeing of human hair is the need for dye systems which avoid any adverse effect on the hair and skin of the user, such as brittle hair, or, irritation of the skin, or, staining (coloring) of the skin.

Thus, it would be desirable to develop a hair coloring composition which exhibits reduced fade, provides improved resistance to wash out during a regular cleansing regimen, can deliver substantially consistent hair color results throughout the hair, which has reduced irritant effect on the skin, which has reduced staining on the skin, which has reduced adverse effects on the hair of the user.

It has now been found that the combination of oxidising agent with one or more oxidative hair coloring agents at a pH below the internal pH of hair, between about pH 1 to about pH 5, in hair coloring compositions can deliver excellent initial hair color in combination with improved color and wash fastness of the hair color over time, desirable color saturation and vividness attributes, reduced hair damage, reduced skin irritation, reduced skin staining and more efficient dyeing. Furthermore, it has been found that the efficacy of color development (color change) from the oxidising agent and oxidative hair coloring agents is improved under the low pH conditions of the present invention. In addition, it has been found that the hair coloring compositions according to the present invention can deliver these excellent hair coloring attributes results with minimal hair damage, at low pH (about 1 to about 5).

It is an object of the present invention to provide storage stable, re-usable, low pH hair coloring compositions which deliver the combination of improved hair coloring attributes, such as, longer lasting color (reduced fade), initial color generation, increased color uptake and color consistency across hair types. Hair types as defined herein means hair of varying age and condition i.e. virgin untreated, grey, permed, bleached etc.

It is a still further object of the present invention to provide storage stable, re-usable, low pH hair coloring compositions which have reduced levels of skin irritation and/or skin staining versus conventional high pH systems and which impart minimal damage to the hair fibres and reduced staining of skin in combination with an acceptable odor.

It is a yet further object of the present invention to provide storage stable, re-usable, low pH hair coloring compositions which exhibit increased efficacy (improved color development). Color development as defined herein, means, the change in the hair color, expressed in terms of Delta E, as defined in the Experimental section herein after. It is a yet further object of the present invention to provide coloring compositions with reduced damage to the skin and/or hair which can deliver equivalent color development (versus conventional high pH systems) in combination with improved washfastness and color consistency while using less dye and/or less of the source of inorganic peroxygen based oxidising agent.

### Hair Coloring Agents

The low pH hair coloring compositions of the present invention include as an essential feature an oxidative hair coloring agent. Such oxidative hair coloring agents are used in combination with the oxidising systems of the present invention to formulate permanent, demi-permanent, semi-permanent or temporary hair dye compositions at low pH.

Permanent hair dye compositions as defined herein are compositions which once applied to the hair are substantially resistant to wash-out. Demi-permanent hair dye compositions as defined herein are compositions which are substantially removed from the hair after up to 24 washes. Semi-permanent hair dye compositions as defined herein are compositions which once applied to the hair are substantially removed from the hair after up to 10 washes. Temporary hair dye compositions as defined herein are compositions which once applied to the hair are substantially removed from the hair after up to 2 washes. These different types of hair coloring compositions can be formulated via the specific combination of oxidant and/or dyes at different levels and ratios. Wash out as defined herein is the process by which hair color is removed from the hair over time during normal hair cleansing regimen. Washfastness as defined herein, means, the resistance of the dyed hair to wash out. Washfastness, as defined herein, can be measured in terms of the relative color change in the dyed hair (Delta E) over several washes (shampoos). Substantial removal of dye from the hair as defined herein means the color change in the dyed hair (Delta E) is greater than about 2 after up to 10 washes.

The concentration of each oxidative hair coloring agent in the low pH coloring compositions according to the present invention is from about 0.001% to about 3% by weight and is preferably from about 0.01% to about 2% by weight.

The total combined level of oxidative hair coloring agents in the compositions according to the present invention is from about 0.001% to about 5%, preferably from about 0.01% to about 4%, more preferably from about 0.1% to about 3%, most preferably from about 0.1% to about 1% by weight.

Typically, in conventional hair coloring compositions the total level of oxidative hair coloring agents present in the composition is in the range of from about 0.2% to about 3.5% by weight. Accordingly, the compositions according to the present invention can display improved hair coloring attributes, such as initial color development and initial color consistency in combination with improved washfastness over time, when compared to conventional, high pH, systems having similar levels of dye. Color consistency, as used herein, means, both the relative predictability of the initial color development and improved color retention over time across different hair types.

The efficacy of the oxidative dyes is improved at low pH such that the compositions of the present invention are valuable for the delivery of good high intensity colors (dark colors) with reduced levels of dye. In particular, good hair coloring results in combination with equivalent color development (versus high pH systems) can be achieved using the inorganic peroxygen oxidising agents of the present invention and substantially less dye versus conventional, high pH, hair coloring compositions.

Thus according to a further aspect of the present invention there is provided a hair coloring composition comprising:

(a) an oxidising agent;

(b) an oxidative hair coloring agent; and

(c) a diluent suitable for application to the hair;

wherein the pH of each of (a) and (b) is in the range of from about 1 to about 6 and wherein the combined mixture of (a) and (b) has a pH in the range of from about 1 to about 5 and wherein both (a) and (b) are capable of being stored at low pH, either separately or once mixed for at least about 1 month at room temperature and wherein the resultant color delivered to the hair (Delta E) is greater than about 75%, preferably greater than about 85%, more preferably greater than about 90% and most preferably greater than about 95% of the total color delivered to the hair (Delta E target) from a mixture of (a) and (b) on mixing.

As herein before described, it has also been found that the combination of oxidising agent with oxidative hair coloring agent at low pH is valuable for the delivery of excellent hair coloring attributes in combination with reduced levels of hair damage, skin irritation and skin staining in combination with an improved odor profile (versus conventional high pH compositions). A further benefit of the low pH coloring compositions according to the present invention is that reduced levels of skin staining can be observed from such compositions, versus conventional, high pH, compositions.

Thus according to a still further aspect of the present invention there is provided a hair coloring composition capable of delivering a light auburn colour to light brown hair having 40% grey comprising:

(a) an oxidising agent;

(b) an oxidative hair coloring agent; and

(c) a diluent suitable for application to the hair;

wherein the pH of each of (a) and (b) is in the range of from about 1 to about 6 and wherein the combined mixture of (a) and (b) has a pH in the range of from about 1 to about 5 and wherein both (a) and (b) are capable of being stored at low pH, either separately or once mixed for at least about 1 month at room temperature and wherein the change in level of skin staining after product application (Delta E) is less than about 4, preferably less than about 3, more preferably less than about 2.7. Light brown hair having 40% grey coverage is defined in terms of L, a, b values as having an ‘L’ value in the range of from about 35 to about 37, an ‘a’ value in the range of from about 4.5 to about 5.5 and a ‘b’ value in the range of from about 11.5 to about 12.7.

Without being limited by any particular theory, it is believed that, these improvements (in respect of reduced skin irritation and/or staining) result from the combination of (a) reduced levels of dyes and low pH; (b) the reduction of paraphenylene diamine (PPD) contact sensitisation at low pH (high levels of PPD have been shown to display contact sensitisation at high pH, but not at low pH); (c) the elimination of the formation of nitrobenzene contact sensitisers (which can occur in high pH compositions); (d) reduced levels of skin staining at low pH versus high pH, and; (e) the reduction in skin irritation and odor negatives as a result of the elimination of ammonia and the use of alternative oxidising agents in the low pH dyeing compositions according to the present invention.

### Oxidative Hair Coloring Processes

Any oxidative hair coloring agent can be used in the compositions according to the present invention. Typically, but without intending to be limited thereby, oxidative hair coloring agents, consist essentially of at least two components, which are collectively referred to as dye forming intermediates (or precursors). Dye forming intermediates can react in the presence of a suitable oxidant to form a colored molecule.

The dye forming intermediates used in oxidative hair colorants include: aromatic diamines, aminophenols, various heterocycles, phenols, napthols and their various derivatives. These dye forming intermediates can be broadly classified as; primary intermediates and secondary intermediates. Primary intermediates, which are also known as oxidative dye precursors, are chemical compounds which become activated upon oxidation and can then react with each other and/or with couplers to form colored dye complexes. The secondary intermediates, also known as color modifiers or couplers, are generally colorless molecules which can form colors in the presence of activated precursors/primary intermediates, and are used with other intermediates to generate specific color effects or to stabilise the color.

Primary intermediates suitable for use in the compositions and processes herein include: aromatic diamines, polyhydric phenols, amino phenols and derivatives of these aromatic compounds (e.g., N-substituted derivatives of the amines, and ethers of the phenols). Such primary intermediates are generally colorless molecules prior to oxidation.

While not wishing to be bound by any particular theory it is proposed herein that the process by which color is generated from these primary intermediates and secondary coupler compounds generally includes a stepwise sequence whereby the primary intermediate can become activated (by oxidation), and then enjoins with a coupler to give a dimeric, conjugated colored species, which in turn can enjoin with another ‘activated’ primary intermediate to produce a trimeric conjugated colored molecule.

### Chemistry of Oxidative Hair Coloration Across pH

While not wishing to be bound by any particular theory, it is generally understood that conventional oxidative dyeing typically occurs between oxidative precursor molecules, oxidative coupler molecules and a peroxygen oxidising agent at high pH (8-10). Typical precursors include 1,4-disubstituted benzene derivatives and typical couplers include 1,2- or 1,3-disubstituted benzene derivatives.

It is generally accepted that the pH within the hair shaft, of human hair, is around pH 5.5 to pH 6 (C. R. Robbins, Chemical and Physical Behaviour of Human Hair, 2nd Ed. p157) and that the hair has an inherent buffering capacity.

Surpisingly it has now been found that, oxidative hair coloration at pH 5 or less with oxidising agent(s) and oxidative hair coloring agent is valuable for the delivery of excellent initial hair color in combination with improved color and wash fastness of the hair color over time, desirable color saturation and vividness attributes, reduced hair damage, reduced skin irritation, reduced skin staining and more efficient dyeing. Furthermore, it has been found that the efficiency of color development (i.e., increased color change) from the inorganic peroxygen oxidising agents and the oxidative hair coloring agents of the present invention is improved under the low pH conditions according to the present invention. In addition, it has been found that the low pH hair coloring compositions according to the present invention can deliver these excellent hair coloring attributes results with minimal hair damage.

It has also been found that at pH levels of less than pH 6, preferably from about pH 1.5 to about 5, more preferably from about 1.8 to about 4.7, most preferably from about 2 to about 4.6, especially from about 2.5 to about 4.5 and most especially from 2.7 to 3.8, further improvements in color development can be acheived.

### Oxidative Dye Precursors

In general terms, oxidative dye primary intermediates include those monomeric materials which, on oxidation, form oligomers or polymers having extended conjugated systems of electrons in their molecular structure. Because of the new electronic structure, the resultant oligomers and polymers exhibit a shift in their electronic spectra to the visible range and appear colored. For example, oxidative primary intermediates capable of forming colored polymers include materials such as aniline, which has a single functional group and which, on oxidation, forms a series of conjugated imines and quinoid dimers, trimers, etc. ranging in color from green to black. Compounds such as p-phenylenediamine, which has two functional groups, are capable of oxidative polymerization to yield higher molecular weight colored materials having extended conjugated electron systems. Oxidative dyes known in the art can be used in the low pH compositions according to the present invention. A representative list of primary intermediates and secondary couplers suitable for use herein is found in Sagarin, “Cosmetic Science and Technology”,” Interscience, Special Ed. Vol. 2 pages 308 to 310. It is to be understood that the primary intermediates detailed below are only by way of example and are not intended to limit the compositions and processes herein.

The typical aromatic diamines, polyhydric phenols, amino phenols, and derivatives thereof, described above as primary intermediates can also have additional substituents on the aromatic ring, e.g. halogen, aldehyde, carboxylic acid, nitro, sulfonic acid and substituted and unsubstituted hydrocarbon groups, as well as additional substituents on the amino nitrogen and on the phenolic oxygen, e.g. substituted and unsubstituted alkyl and aryl groups.

Examples of suitable aromatic diamines, amino phenols, polyhydric phenols and derivatives thereof, respectively, are compounds having the general formulas (I), (II) and (III) below:

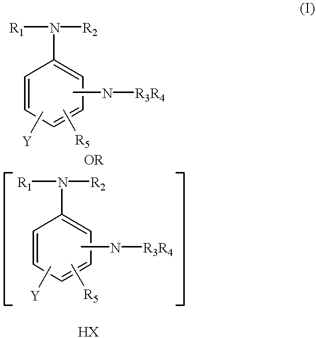

wherein Y is hydrogen, halogen, (e.g. fluorine, chlorine, bromine or iodine), nitro, amino, hydroxyl,

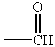

—COOM or —SO₃M (where M is hydrogen or an alkali or alkaline earth metal, ammonium, or substituted ammonium wherein one or more hydrogens on the ammonium ion is replaced with a 1 to 3 carbon atom alkyl or hydroxyalkyl radical), wherein R₁, R₂, R₃ and R₄ are the same or different from each other and are selected from the group consisting of hydrogen, C₁ to C₄ alkyl or alkenyl and C₆ to C₉ aryl, alkaryl or aralkyl, and R₅ is hydrogen, C₁ to C₄ unsubstituted or substituted alkyl or alkenyl wherein the substituents are selected from those designated as Y, above, or C₆ to C₉ unsubstituted or substituted aryl, alkaryl or aralkyl wherein the substituents are selected from those defined as Y, above. Since the precursors of formula (I) are amines, they can be used herein in the form of peroxide-compatible salts, as noted, wherein X represents peroxide-compatible anions of the type herein before detailed. The general formula of the salt indicated is to be understood to encompass those salts having mono-, di-, and tri-negative anions.

Specific examples of formula (I) compounds are: o-phenylenediamine, m-phenylenediamine, p-phenylenediamine, 2-chloro-p-phenylenediamine, 2-iodo-p-phenylenediamine, 4-nitro-o-phenylenediamine, 2-nitro-p-phenylenediamine, 1,3,5-triaminobenzene, 2-hydroxy-p-phenylenediamine, 2,4-diaminobenzoic acid, sodium 2,4-diaminobenzoate, calcium di-2,4-diaminobenzoate, ammonium 2,4-diaminobenzoate, trimethylammonium 2,4-, diaminobenzoate, tri-(2-hydroxyethyl)ammonium 2,4-diaminobenzoate, 2,4-diaminobenzaldehyde carbonate, 2,4-diaminobenzensulfonic acid, potassium 2,4-diaminobenzenesulfonate, N,N-diisopropyl-p-, phenylenediamine bicarbonate, N,N-dimethyl-p-phenylenediamine, N-ethyl-N′-(2-propenyl)-p-phenylenediamine, N-phenyl-p-phenylenediamine, N-phenyl-N-benzyl-p-phenylenediamine, N-ethyl-N′-(3-ethylphenyl)-p-phenylenediamine, 2,4-toluenediamine, 2-ethyl-p-phenylenediamine, 2-(2-bromoethyl)-p-phenylenediamine, 2-phenyl-p-phenylenediamine laurate, 4-(2,5-diaminophenyl)benzaldehyde, 2-benzyl-p-phenylenediamine acetate, 2-(4-nitrobenzyl)-p-phenylenediamine, 2-(4-methylphenyl)-p-phenylenediamine, 2-(2,5-diaminophenyl)-5-methylbenzoic acid, methoxyparaphenylenediamine, dimethyl-p-phenylenediamine, 2,5-dimethylpara-phenylenediamine, 2-methyl-5-methoxy-para-phenylenediamine, 2,6-methyl-5-methoxy-para-phenylenediamine, 3-methyl-4-amino-N,N-diethylaniline, N,N-bis(β-hydroxyethyl)-paraphenylenediamine, 3-methyl-4-amino-N,N-bis(β-hydroxyethyl)aniline, 3-chloro-4-amino-N,N-bis(β-hydroxyethyl)aniline, 4-amino-N-ethyl-N-(carbamethyl)aniline, 3-methyl-4-amino-N-ethyl-N-(carbamethyl)aniline, 4-amino-N-ethyl-(β-piperidonoethyl)aniline, 3-methyl-4-amino-N-ethyl-(β-piperidonoethyl)aniline, 4-amino-N-ethyl-N-(β-morpholinoethyl)aniline, 3-methyl-4-amino-N-ethyl-N-(β-morpholinoethyl)aniline, 4-amino-N-ethyl-N-(β-acetylaminoethyl)aniline, 4-amino-N-(β-methoxyethyl)aniline, 3-methyl-4-amino-N-ethyl-N-(β-acetylaminoethyl)aniline, 4-amino-N-ethyl-N-(β-mesylaminoethyl)aniline, 3-methyl-4-amino-N-ethyl-N-(β-mesylaminoethyl)aniline, 4-amino-N-ethyl-N-(β-sulphoethyl)aniline, 3-methyl-4-amino-N-ethyl-N-(β-sulphoethyl)aniline, N-(4-aminophenyl)morpholine, N-(4-aminophenyl)piperidine, 2,3-dimethyl-p-phenylenediamine, isopropyl-p-phenylenediamine, N,N-bis-(2-hydroxyethyl)-p-phenylenediamine sulphate.

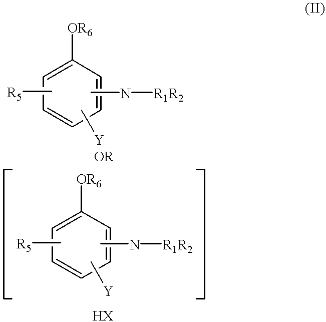

where X and Y are the same as in formula (I), R₁ and R₂ can be the same or different from each other and are the same as in formula (I), R₅ is the same as in formula (I) and R₆ is hydrogen or C₁ to C₄ substituted or unsubstituted alkyl or alkenyl wherein the substituents are selected from those defined as Y in formula (I).

Specific examples of formula (II) compounds are: o-aminophenol, m-aminophenol, p-aminophenol, 2-iodo-p-aminophenol, 2-nitro-p-aminophenol, 3,4-dihydroxyaniline, 3,4-diaminophenol, chloroacetate, 2-hydroxy-4-aminobenzoic acid, 2-hydroxy-4-aminobenzaldehyde, 3-amino-4-hydroxybenzenesulfonic acid, N,N-diisopropyl-p-aminophenol, N-methyl-N-(1-propenyl)-p-aminophenol, N-phenyl-N-benzyl-p-aminophenol sulphate, N-methyl-N-(3-ethylphenyl)-p-aminophenol, 2-nitro-5-ethyl-p-aminophenol, 2-nitro-5-(2-bromoethyl)-p-aminophenol, (2-hydroxy-5-aminophenyl)acetaldehyde, 2-methyl-p-aminophenol, (2-hydroxy-5-aminophenyl)acetic acid, 3-(2-hydroxy-5-aminophenyl)-1-propene, 3-(2-hydroxy-5-aminophenyl)-2-chloro-1-propene, 2-phenyl-p-aminophenol palmitate, 2-(4-nitrophenyl)-p-aminophenol, 2-benzyl-p-aminophenol, 2-(4-chlorobenzyl-p-aminophenol perchlorate, 2-(4-methylphenyl)-p-aminophenol, 2-(2-amino-4-methylphenyl)-p-aminophenol, p-methoxyaniline, 2-bromoethyl-4-aminophenyl ether phosphate, 2-nitroethyl-4-aminophenyl ether bromide, 2-aminoethyl-4-aminophenyl ether, 2-hydroxyethyl-4-aminophenyl ether, (4-aminophenoxy)acetaldehyde, (4-aminophenoxy)acetic acid, (4-aminophenoxy)methanesulfonic acid, 1-propenyl-4-aminophenyl ether isobutyrate, (2-chloro)-1-propenyl-4-aminophenyl ether, (2-nitro)-1-propenyl-4-aminophenyl ether, (2-amino)-propenyl-4-aminophenyl ether, (2-hydroxy)-1-propenyl-4-aminophenyl ether, N-methyl-p-aminophenol, 3-methyl-4-aminophenol, 2-chloro-4-aminophenol, 3-chloro-4-aminophenol, 2,6-dimethyl-4-aminophenol, 3,5-dimethyl-4-aminophenol, 2,3-dimethyl-4-aminophenol, 2,5-dimethyl-4-aminophenol, 2-hydroxymethyl-4-aminophenol, 3-hydroxymethyl-4-aminophenol.

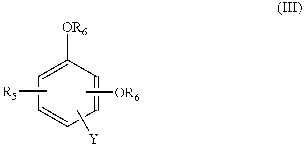

where Y, R₅ and R₆ are as defined above in formula (II).

Specific examples of formula (III) compounds are: o-hydroxyphenol (catechol), m-hydroxyphenol (resorcinol), p-hydroxyphenol (hydroquinone), 4-methoxyphenol, 2-methoxyphenol, 4-(2-chloroethoxy)phenol, 4-(2-propenoxy)phenol, 4-(3-chloro-2-propenoxy)phenol, 2-chloro-4-hydroxyphenol (2-chlorohydroquinone), 2-nitro-4-hydroxyphenol(2-nitrohydroquinone), 2-amino-4-hydroxyphenol, 1,2,3-trihydroxybenzene (pyrogallol), 2,4-dihydroxybenzaldehyde, 3,4-dihydoxybenzoic acid, 2,4-dihydroxybenzenesulfonic acid, 3-ethyl-4-hydroxyphenol, 3-(2-nitroethyl)-4-hydroxyphenol, 3-(2-propenyl)-4-hydroxyphenol, 3-(3-chloro-2-propenyl)-4-hydroxyphenol, 2-phenyl-4-hydroxyphenol, 2-(4-chlorophenyl)-4-hydroxyphenol, 2-benzyl-4-hydroxyphenol, 2-(2-nitrophenyl)-4-hydroxyphenol, 2-(2-methylphenyl)-4-hydroxyphenol, 2-(2-methyl-4-chlorophenyl)-4-hydroxyphenol, 3-methoxy-4-hydroxy-benzaldehyde, 2-methoxy-4-(1-propenyl)phenol, 4-hydroxy-3-methoxycinnamic acid, 2,5-dimethoxyaniline, 2-methylresorcinol, alpha napthol and salts thereof.

Secondary coupling compounds (color modifiers), such as those detailed hereinafter, are preferably used in conjunction with the primary intermediates herein and are thought to interpose themselves in the colored polymers during their formation and to cause shifts in the electronic spectra thereof, thereby resulting in slight color changes.

Secondary coupling compounds which are suitable for inclusion in the coloring compositions and processes herein before described include certain aromatic amines and phenols and derivatives thereof which do not produce color singly, but which modify the color, shade or intensity of the colors developed by the primary oxidized dye intermediates. Certain aromatic amines and phenolic compounds, and derivatives thereof, including some aromatic diamines and polyhydric phenols of the types described by formulas (I), (II) and (III) above, but which are well known in the art not to be suitable primary intermediates, are suitable as couplers herein. Polyhydric alcohols are also suitable for use as couplers herein.

The aromatic amines and phenols and derivatives described above as couplers can also have additional substituents on the aromatic ring, e.g., halogen, aldehyde, carboxylic acid, nitro, sulfonyl and substituted and unsubstituted by hydrocarbon groups, as well as additional substituents on the amino nitrogen, or phenolic oxygen, e.g. substituted and unsubstituted alkyl and aryl groups. Again, peroxide-compatible salts thereof are suitable for use herein.

Examples of aromatic amines, phenols and derivatives thereof are compounds of the general formulas (IV) and (V) below:

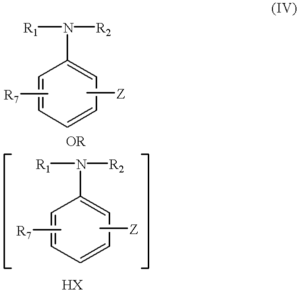

wherein Z is hydrogen, C₁ and C₃ alkyl, halogen (e.g. fluorine, chlorine, bromine or iodine) nitro,

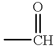

—COOM or SO₃M, (where M is hydrogen or an alkali or alkaline earth metal, ammonium or substituted ammonium wherein one or more hydrogens on the ammonium ion is replaced with a 1 to 3 carbon atom alkyl or hydroxyalkyl radical), wherein R₁ and R₂ are the same or different and are selected from the group consisting of hydrogen, C₁ to C₄ alkyl or alkenyl and C₆ to C₉ aryl, alkaryl or aralkyl and R₇ is hydrogen, C₁ to C₄ unsubstituted or substituted alkyl or alkenyl wherein the substituents are selected from those designated as Z above or C₆ to C₉ unsubstituted or substituted aryl, alkaryl or aralkyl wherein the substituents are selected from those defined as Z above and wherein X is as defined in formula (I).

Specific examples of formula (IV) compounds are: aniline, p-chloroaniline, p-fluoroaniline, p-nitroaniline, p-aminobenzaldehyde, p-aminobenzoic acid, sodium-p-aminobenzoate, lithium-p-aminobenzoate, calcium di-p-aminobenzoate, ammonium-p-aminobenzoate, trimethylammonium-p-aminobenzoate, tri(2-hydroxyethyl)-p-aminobenzoate, p-aminobenzenesulfonic acid, potassium p-aminobenzenesulfonate, N-methylaniline, N-propyl-N-phenylaniline, N-methyl-N-2-propenylaniline, N-benzylaniline, N-(2-ethylphenyl)aniline, 4-methylaniline, 4-(2-bromoethyl)aniline, 2-(2-nitroethyl)aniline, (4-aminophenyl)acetaldehyde, (4-aminophenyl)acetic acid, 4-(2-propenyl)aniline acetate, 4-(3-bromo-2-propenyl)aniline, 4-phenylaniline chloroacetate, 4-(3-chlorophenyl)aniline, 4-benzylaniline, 4-(4-iodobenzyl)aniline, 4-(3-ethylphenyl)aniline, 4-(2-chloro-4-ethylphenyl)aniline.

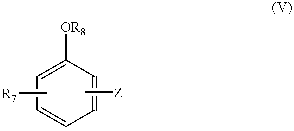

wherein Z and R₇ are defined as in formula (IV) and R₈ is hydrogen or C₁ to C₄ substituted or unsubstituted alkyl or alkenyl wherein the substituents are selected from those defined as Z in formula (IV).

Specific examples of formula (V) compounds are: phenol, p-chlorophenol, p-nitrophenol, p-hydroxybenzaldehyde, p-hydroxybenzoic acid, p-hydroxybenzenesulfonic acid, ethylphenyl ether, 2-chloroethylphenyl ether, 2-nitroethylphenyl ether, phenoxyacetaldehyde, phenoxyacetic acid, 3-phenoxy-1-propene, 3-phenoxy-2-nitro-1-propene, 3-phenoxy-2-bromo-1-propene, 4-propylphenol, 4-(3-bromopropyl)phenol, 2-(2-nitroethyl)phenol, (4-hydroxyphenyl)acetaldehyde, (4-hydroxyphenyl)acetic acid, 4-(2-propenyl)phenol, 4-phenylphenol, 4-benzylphenol, 4-(3-fluoro-2-propenyl)phenol, 4-(4-chlorobenzyl)phenol, 4-(3-ethylphenyl)phenol, 4-(2-chloro-3-ethylphenyl)phenol, 2,5-xylenol, 2,5-diaminopyridine, 2-hydroxy-5-aminopyridine, 2-amino-3-hydroxy pyridine, tetraaminopyrimindine, 1,2,4-trihydroxybenzene, 1,2,4-trihydroxy-5-(C₁-C₆-alkyl)benzene, 1,2,3-trihydroxybenzene, 4-aminoresorcinol, 1,2-dihydroxybenzene, 2-amino-1,4-dihydroxybenzene, 2-amino-4-methoxy-phenol, 2,4-diaminophenol, 3-methoxy-1,2-dihydroxy-benzene, 1,4-dihydroxy-2-(N,N-diethylamino)benzene, 2,5-diamino-4-methoxy-1-hydroxybenzene, 4,6-dimethoxy-3-amino-1-hydroxybenzene, 2,6-dimethyl-4-[N-(p-hydroxyphenyl)amino]-1-hydroxybenzene, 1,5-diamino-2-methyl-4-[N-(p-hydroxyphenyl)amino]benzene and salts thereof.

Additional primary intermediates suitable for use herein include catechol species and in particular catechol “dopa” species which includes dopa itself as well as homologs, analogs and derivatives of DOPA. Examples of suitable cachetol species include cysteinyl dopa, alpha alkyl dopa having 1 to 4, preferably 1 to 2 carbon atoms in the alkyl group, epinephrine and dopa alkyl esters having 1 to 6, preferably 1 to 2 carbon atoms in the alkyl group.

In general suitable catechols are represented by formula (VI) below:

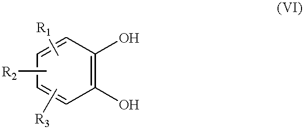

wherein R₁, R₂ and R₃, which may be the same or different, are electron donor or acceptor substutuents selected from H, lower (C₁-C₆) alkyl, OH, OR, COOR, NHCOR, CN, COOH, Halogen, NO₂, CF₃, SO₃H or NR₄R₅, with the proviso that only one of the R₁, R₂ or R₃ can be CN, COOH, halogen, NO₂, CF₃ or SO₃H: R₄ and R₅, which may be the same or different, are H, lower (C₁-C₆) alkyl or substituted lower (C₁-C₆) alkyl in which the substituent may be OH, OR, NHCOR₆, NHCONH₂, NHCO₂R₆, NHCSNH₂, CN, COOH, SO₃H, SO₂NR₆, SO₂R₆ or CO₂R₆; R₆ is lower (C₁-C₆) alkyl, lower (C₁-C₆) hydroxyalkyl phenyl linked to the nitrogen by an alkylene chain, phenyl or substituted phenyl with the substituent defined as R₁, and R is C₁-C₆ alkyl or C₁-C₆ hydroxyalkyl.

Also included herein are oxidative hair coloring agents of the formula:

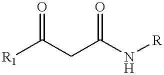

wherein: R₁=substituted or unsubstituted benzene ring, tertiary-butyl, etc.; R=substituted or unsubstituted benzene ring and the formula:

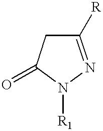

wherein R=aminoalkyl, amidoalkyl, aminobenzene (substituted or unsubstituted), amidobenzene (substituted or unsubstituted), alkyl, substituted or unsubstituted benzene ring; R₁=substituted or unsubstituted benzene ring.

The primary intermediates can be used herein alone or in combination with other primary intermediates, and one or more can be used in combination with one or more couplers. The choice of primary intermediates and couplers will be determined by the color, shade and intensity of coloration which is desired. There are nineteen preferred primary intermediates and couplers which can be used herein, singly or in combination, to provide dyes having a variety of shades ranging from ash blonde to black; these are: pyrogallol, resorcinol, p-toluenediamine, p-phenylenediamine, o-phenylenediamine, m-phenylenediamine, o-aminophenol, p-aminophenol, 4-amino-2-nitrophenol, nitro-p-phenylenediamine, N-phenyl-p-phenylenediamine, m-aminophenol, 2-amino-3-hydroxypyridine, 1-napthol, N,N bis (2-hydroxyethyl)p-phenylenediamine, 4-amino-2-hydroxytoluene, 1,5-dihydroxynapthalene, 2-methyl resorcinol and 2,4-diaminoanisole. These can be used in the molecular form or in the form of peroxide-compatible salts, as detailed above.

The primary intermediates and coupling compounds as aforementioned herein may be combined to deliver a wide variety of colors to the hair. The hair colors can vary by both depth of color and intensity of color. As hereinbefore described the compositions according to the present invention are valuable for the provision of high intensity colors. Intensity of color as defined herein means the quantity of color compound formed on and retained in the hair. In general, high intensity as defined herein means dark or deep colors such as dark red, dark brown or black etc. In accordance, with the above it is possible to formulate hair colors of varying color intensity by adjusting the initial levels of each of the oxidative dyeing materials.

For example low intensity colors such as natural blond to light brown hair shades generally comprise from about 0.001% to about 5%, preferably from about 0.1% to about 2%, more preferably from about 0.2% to about 1% by weight of coloring composition of total oxidative dyeing agents and may be achieved by the combination of primary intermediates such as 1,4-diamino-benzene, 2,5-diamino toluene, 2,5-diamino-anisole, 4-aminophenol, 2,5-diamino-benzyl alcohol and 2-(2′,5′-diamino)phenylethanol with couplers such as resorcinol, 2-methyl resorcinol or 4-chloro resorcinol.

Similarly combination of the above primary intermediates with couplers, such as, 5-amino-2-methyl phenol and 1,3-diamino-benzene derivatives such as 2,4-diamino-anisole at levels of from about 0.5% to about 1% of total dyeing agents can lead to medium intensity red colors. High intensity colors such as blue to blue-violet hair shades can be produced by the combination of the above primary intermediates with couplers such as 1,3-diamino-benzene or its derivatives such as 2,5-diamino-toluene at levels of from about 1% to about 6% by weight of composition of total dyeing agents. Black hair colors can be obtained by combining the aforementioned primary intermediates with couplers such as 1,3-diaminobenzene or its derivatives

However considerations have been raised against the physiological compatibility of para-amino phenol which is commonly used to impart red colors to the hair. Similarly, the physiological compatibility of some of the agents favoured for the production of black color such as paraphenylene diamine (PPD) has been called into question. Thus a need exists for oxidative hair coloring compositions which have an improved safety profile and in particular oxidative hair compositions for the delivery of dark colors i.e. high color intensity dyes, which have an improved safety profile. As discussed herein before, the low pH compositions of the present invention provide excellent hair coloring attributes in combination with reduced levels of hair damage and skin staining and/or irritation.

As such the compositions according to the present invention are valuable for the delivery of improved hair condition attributes in combination with good initial color development and consistency and improved wash fastness over time in addition to having reduced levels of hair damage and skin irritation and/or staining.

### Non-Oxidative and Other Dyes

The hair coloring compositions of the present invention may, in addition to the essential oxidative hair coloring agents, optionally include non-oxidative and other dye materials. Optional non-oxidative and other dyes suitable for use in the hair coloring compositions and processes according to the present invention include both semi-permanent, temporary and other dyes. Non-oxidative dyes as defined herein include the so-called ‘direct action dyes’, metallic dyes, metal chelate dyes, fibre reactive dyes and other synthetic and natural dyes. Various types of non-oxidative dyes are detailed in: ‘Chemical and Physical Behaviour of Human Hair’ 3rd Ed. by Clarence Robbins (pp250-259); ‘The Chemistry and Manufacture of Cosmetics’. Volume IV. 2nd Ed. Maison G. De Navarre at chapter 45 by G. S. Kass (pp841-920); ‘cosmetics: Science and Technology’ 2nd Ed., Vol. II Balsam Sagarin, Chapter 23 by F. E. Wall (pp 279-343); ‘The Science of Hair Care’ edited by C. Zviak, Chapter 7 (pp 235-261) and .‘Hair Dyes’, J. C. Johnson, Noyes Data Corp., Park Ridge, U.S.A. (1973), (pp 3-91 and 113-139).

Direct action dyes which do not require an oxidative effect in order to develop the color, are also designated hair tints and have long been known in the art. They are usually applied to the hair in a base matrix which includes surfactant material. Direct action dyes include nitro dyes such as the derivatives of nitroamino benzene or nitroaminophenol; disperse dyes such as nitroaryl amines, aminoanthraquinones or azo dyes; anthraquinone dyes, naphthoquinone dyes; basic dyes such as Acridine Orange C.I. 46005.

Nitrodyes are added to dyeing compositions to enhance colour of colorant and to add suitable aesthetic colour to the dye mixture prior to application.

Further examples of direct action dyes include the Arianor dyes basic brown 17, C.I.(color index)—no. 12,251; basic red 76, C.I.—12,245; basic brown 16, C.I.—12,250; basic yellow 57, C.I.—12,719 and basic blue 99, C.I.—56,059 and further direct action dyes such as acid yellow 1, C.I.—10,316 (D&C yellow no.7); acid yellow 9, C.I.—13,015; basic violet C.I.—45,170; disperse yellow 3, C.I.—11,855; basic yellow 57, C.I.—12,719; disperse yellow 1, C.I.—10,345; basic violet 1, C.I.—42,535, basic violet 3, C.I.—42,555; greenish blue, C.I.—42090 (FD&C Blue no.1); yellowish red, C.I.—14700 (FD&C red no.4); yellow, C.I.19140 (FD&C yellow no5); yellowish orange, C.I.15985 (FD&C yellow no.6); bluish green, C.I.42053 (FD&C green no.3); yellowish red, C.I.16035 (FD&C red no.40); bluish green, C.I.61570 (D&C green no.3); orange, C.I.45370 (D&C orange no.5); red, C.I.15850 (D&C red no.6); bluish red, C.I.15850 (D&C red no.7); slight bluish red, C.I.45380 (D&C red no.22); bluish red, C.I.45410 (D&C red no.28); bluish red, C.I.73360 (D&C red no.30); reddish purple, C.I.17200 (D&C red no.33); dirty blue red, C.I.15880 (D&C red no.34); bright yellow red, C.I.12085 (D&C red no.36); bright orange, C.I.15510 (D&C orange no.4); greenish yellow, C.I.47005 (D&C yellow no.10); bluish green, C.I.59040 (D&C green no.8); bluish violet, C.I.60730 (Ext. D&C violet no.2); greenish yellow, C.I.10316 (Ext. D&C yellow no.7);

Fibre reactive dyes include the Procion (RTM), Drimarene (RTM), Cibacron (RTM), Levafix (RTM) and Remazol (RTM) dyes available from ICI, Sandoz, Ciba-Geigy, Bayer and Hoechst respectively.

Natural dyes and vegetable dyes as defined herein include henna (Lawsonia alba), camomile (Matricaria chamomilea or Anthemis nobilis), indigo, logwood and walnut hull extract.

Temporary hair dyes, or hair coloring rinses, are generally comprised of dye molecules which are too large to diffuse into the hair shaft and which act on the exterior of the hair. They are usually applied via a leave-in procedure in which the dye solution is allowed to dry on the hair surface. As such these dyes are typically less resistant to the effects of washing and cleaning the hair with surface active agents and are washed off of the hair with relative ease. Any temporary hair dye may suitably be used in the compositions of the invention and examples of preferred temporary hair dyes are illustrated below.

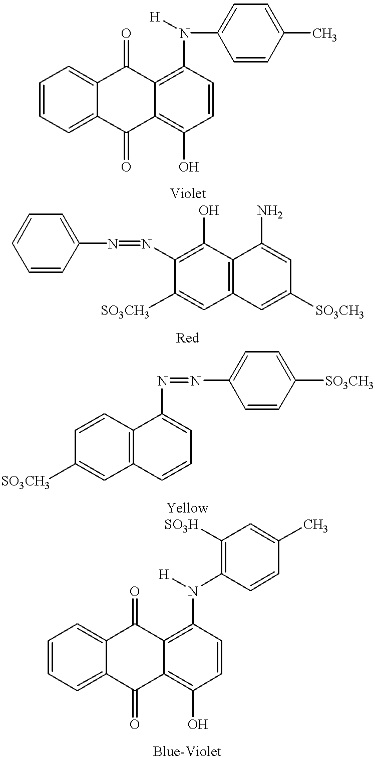

Semi-permanent hair dyes are dyes which are generally smaller in size and effect to temporary hair rinses but are generally larger than permanent (oxidative) dyes. Typically, semi-permanent dyes act in a similar manner to oxidative dyes in that they have the potential to diffuse into the hair shaft. However, semi-permanent dyes are generally smaller in size than the aforementioned conjugated oxidative dye molecules and as such are pre-disposed to gradual diffusion out of the hair again. Simple hair washing and cleaning action will encourage this process and in general semi-permanent dyes are largely washed out of the hair after about 5 to 8 washes. Any semi-permanent dye system may be suitably used in the compositions of the present invention. Suitable semi-permanent dyes for use in the compositions of the present invention are HC Blue 2, HC Yellow 4, HC Red 3, Disperse Violet 4, Disperse Black 9, HC Blue 7, HC Yellow 2, Disperse Blue 3, Disperse violet 1 and mixtures thereof. Examples of semi-permanent dyes are illustrated below:

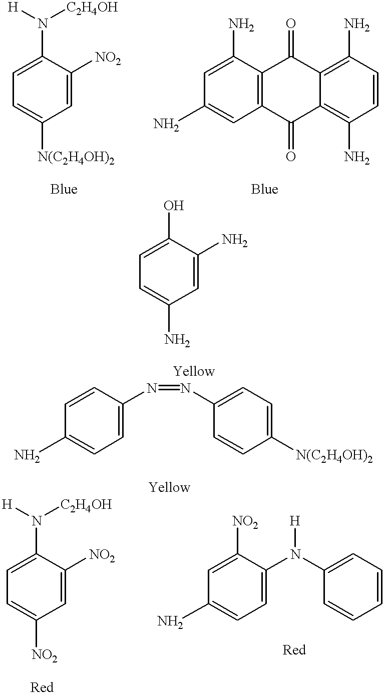

Typical semi-permanent dye systems incorporate mixtures of both large and small color molecules. As the size of the hair is not uniform from root to tip the small molecules will diffuse both at the root and tip, but will not be retained within the tip, while the larger molecules will be generally only be able to diffuse into the ends of the hair. This combination of dye molecule size is used to help give consistent color results from the root to the tip of the hair both during the initial dyeing process and during subsequent washing.

### Buffering Agents

The coloring compositions of the present invention have a pH in the range of from about 1 to about 6, preferably 1.5 to about 5, more preferably from about 1.8 to about 4.7, most preferably from about 2 to about 4.6, especially from about 2.5 to about 4.5 and most especially from 2.7 to 3.8.

As herein before described the pH of the preferred coloring compositions of the present invention are maintained within the desired pH range via the action of the inorganic peroxygen oxidising agent. However, if so desired, the compositions may contain one or more optional buffering agents and/or hair swelling agents (HSAs). Several different pH modifiers can be used to adjust the pH of the final composition or any constituent part thereof.

This pH adjustment can be effected by using well known acidifying agents in the field of treating keratinous fibres, and in particular human hair, such as inorganic and organic acids such as hydrochloric acid, tartaric acid, citric acid, succinic acid, phosphoric acid and carboxylic or sulphonic acids such as ascorbic acid, acetic acid, lactic acid, sulphuric acid, formic acid, ammonium sulphate and sodium dihydrogenphosphate/phosphoric acid, disodium hydrogenphosphate/phosphoric acid, potassium chloride/hydrochloric acid, potassium dihydrogen phthalate/hydrochloric acid, sodium citrate/hydrochloric acid, potassium dihydrogen citrate/hydrochloric acid, potassium dihydrogencitrate/citric acid, sodium citrate/citric acid, sodium tartarate/tartaric acid, sodium lactate/lactic acid, sodium acetate/acetic acid, disodium hydrogenphosphate/citric acid and sodium chloride/glycine/hydrochloric acid, succinic acid and mixtures thereof.

Examples of alkaline buffering agents are ammonium hydroxide, ethylamine, dipropylamine, triethylamine and alkanediamines such as 1,3-diaminopropane, anhydrous alkaline alkanolamines such as, mono or di-ethanolamine, preferably those which are completely substituted on the amine group such as dimethylaminoethanol, polyalkylene polyamines such as diethylenetriamine or a heterocyclic amine such as morpholine as well as the hydroxides of alkali metals, such as sodium and potassium hydroxide, hydroxides of alkali earth metals, such as magnesium and calcium hydroxide, basic amino acids such as L-argenine, lysine, alanine, leucine, iso-leucine, oxylysine and histidine and alkanolamines such as dimethylaminoethanol and aminoalkylpropanediol and mixtures thereof. Also suitable for use herein are compounds that form HCO₃— by dissociation in water (hereinafter referred to as ‘ion forming compounds’). Examples of suitable ion forming compounds are Na₂CO₃, NaHCO₃, K₂CO₃, (NH₄)₂CO₃, NH₄HCO₃, CaCO₃ and Ca(HCO₃) and mixtures thereof.

Preferred for use herein as buffering agents are organic and inorganic acids having a first pKa below about pH 6, and their conjugate bases. As defined herein, first pKa means, the negative logarithm (to the base 10) of the equilibrium constant, K, where K is the acid dissociation constant. Suitable organic and inorganic acids for use herein are: aspartic, maleic, tartaric, glutamic, glycolic, acetic, succinic, salycilic, formic, benzoic, malic, lactic, malonic, oxalic, citric, phosphoric acid and mixtures thereof. Particularly preferred are acetic, succinic, salycilic and phosphoric acids and mixtures thereof.

The low pH coloring compositions according to the present invention, when a form of intended use, may, as will be described later herein, may be comprised of a final solution containing both oxidising agent and an oxidative hair coloring agent which have been admixed prior to application to the hair or a single component system. Additional, optional materials may be present either in combination with the oxidising agent/coloring agent mixture or as separately packaged units. As such, the compositions according to the present invention may comprise coloring kits of a number of separate components.

In oxidising and coloring kits comprising a portion of inorganic peroxygen oxidising agent, which may be present in either solid or liquid form, such as hydrogen peroxide, a buffering agent solution can be used to stabilise hydrogen peroxide. Since hydrogen peroxide is stable in the pH range from 2 to 4, it is necessary to use a buffering agent having a pH within this range. Dilute acids are suitable hydrogen peroxide buffering agents.

In oxidising and coloring kits comprising an oxidising agent (which may be in solid or liquid form) in combination with one or more coloring agents, a buffering agent capable of maintaining a solution pH in the range of from about 1 to about 6, preferably from about 1.5 to about 5, more preferably from about 1.8 to about 4.7, most preferably from about 2 to about 4.6, especially from about 2.5 to about 4.5 and most especially from 2.7 to 3.8. As such it is necessary to use a buffering agent having a pH within this range.

### Catalyst

The coloring compositions herein may optionally contain a transition metal containing catalyst for the peroxygen oxidising agents and the, optional, preformed peroxy acid oxidising agent(s). One suitable type of catalyst is a catalyst system comprising a heavy metal cation of defined bleach catalytic activity, such as copper, iron or manganese cations, an auxiliary metal cation having little or no bleach catalytic activity, such as zinc or aluminium cations, and a sequestrant having defined stability constants for the catalytic and auxiliary metal cations, particularly ethylenediaminetetraacetic acid, ethylenediaminetetra (methylenephosphonic acid) and water-soluble salts thereof. Such catalysts are disclosed in U.S. Pat. No. 4,430,243.

Other types of suitable catalysts include the manganese-based complexes disclosed in U.S. Pat. No. 5,246,621 and U.S. Pat. No. 5,244,594. Preferred examples of these catalysts include Mn^{IV}₂(u-O)₃(1,4,7-trimethyl-1,4,7-

What is claimed is:

1. A storage stable, re-usable hair coloring composition comprising:
(a) an oxidising agent; and
(b) an oxidative hair coloring agent;
wherein the pH of each of (a) and (b) is in the range of from about 1 to about 6 and wherein the combined mixture of (a) and (b) has a pH in the range of from about 1 to about 5 and wherein both (a) and (b) are capable of being stored at low pH, either separately, or, once mixed, for at least 1 month at room temperature and wherein the resultant color delivered to the hair (Delta E) is up to about 75%, preferably up to about 85%, more preferably up to about 90% and most preferably up to about 95% of the total color delivered to the hair (Delta E) from a mixture of (a) and (b) on mixing.

2. A composition according to claim 1 wherein the pH of the composition is in the range of from about 1.5 to about 5, more preferably from about 1.8 to about 4.7, most preferably from about 2.5 to about 4.5 and especially from about 2.7 to to about 3.8

3. A composition according to claim 1 or 2 wherein the oxidising agent is selected from inorganic peroxygen oxidising agents, preformed or organic peroxyacid oxidising agents, organic peroxide oxidising agents and mixtures thereof.

4. A composition according to any of claims 1 to 3 wherein the oxidising agent is an inorganic peroxygen oxidising agent present at a level of from about 0.01% to about 3%, preferably from about 0.01% to about 2%, more preferably from about 0.01% to about 1%, most especially from about 0.01% to about 0.7% by weight of the total composition.

5. A composition according to any of claims 1 to 4 wherein the inorganic peroxygen oxidising agent is hydrogen peroxide.

6. A composition according to any of claims 1 to 5 wherein the total combined level of oxidative hair coloring agent is from about 0.001% to about 5%, preferably from about 0.01% to about 4%, more preferably from about 0.1% to about 3%, most preferably from about 0.1% to about 1% by weight.

7. A composition according to any of claims 1 to 6 wherein each oxidative hair coloring agent is present at a level of from about 0.001% to about 3%, preferably from about 0.01% to about 2% by weight.

8. A composition according to any of claims 1 to 7 additionally comprising one or more buffering agents, preferably an organic and/or organic acid having a first pKa below pH 6, selected from: aspartic, maleic, tartaric, glutamic, glycolic, acetic, succinic, salycilic, formic, benzoic, malic, lactic, malonic, oxalic, citric, phosphoric acid and mixtures thereof, more preferably selected from acetic, succinic, salycilic and phosphoric acids and mixtures thereof.

9. A composition according to any of claims 1 to 9 additionally comprising one or more surfactants selected from anionic, nonionic, cationic, zwitterionic, amphoteric surfactants and mixtures thereof.

10. A composition according to any of claims 1 to 9 additionally comprising thickening agents, stabilisers, antioxidants and/or any other cosmetically acceptable material.

11. Use of a composition according to any of claims 1 to 10 for coloring human or animal hair.

13. A method for coloring hair wherein a hair coloring mixture is present in a single package suitable for direct application to the hair wherein the hair coloring mixture comprises:
(a) an oxidising agent; and
(b) an oxidative hair coloring agent;
wherein the combined mixture of (a) and (b) has a pH in the range of from about 1 to about 5 and wherein the resultant color delivered to the hair (Delta E), after at least about 1 month of storage at room temperature, is up to about 75%, preferably up to about 85%, more preferably up to about 90% and most preferably up to about 95% of the total color delivered to the hair (Delta E) from a mixture of (a) and (b) on mixing.

14. A hair coloring kit comprising an individually packaged oxidising component and an individually packaged coloring component, wherein the oxidising component comprises an aqueous solution of an inorganic peroxygen oxidising agent at a level of from about 0.01% to about 3% by weight having a pH in the range of from about 1 to about 6 and the coloring component comprises an oxidative hair coloring agent, capable of delivering a solution pH in the range of from about 1 to about 6, at a level of from about 0.001% to about 5% by weight of the composition and wherein the pH of the combined oxidising agent and coloring components is in the range of from about 1 to about 5.

15. Use of a hair coloring kit according to claim 15 for the oxidising and coloring of hair wherein the oxidising agent, oxidative hair coloring agents and additional agents are admixed prior to application to the hair.

16. A method for coloring hair wherein a hair coloring composition is present as separately packaged components (a) and (b) and wherein the hair coloring composition comprises:
(a) from about 0.0003 moles (per 100 g of composition) to less than about 0.2 moles (per 100 g of composition) of an oxidising agent wherein the pH of (a), is in the range of from about 1 to about 6; and
(b) an oxidative hair coloring agent wherein the pH of (b), is in the range of from about 1 to about 6;
wherein each of (a) and (b), when in the form of intended use, are stable at pHs in the range of from about 1 to about 6 and wherein the combined hair coloring mixture of (a) and (b) is stable over time and has a pH in the range of from about 1 to about 5 and wherein the resultant color delivered to the hair (Delta E), after at least about 1 month of storage at room temperature, is up to about 75%, preferably up to about 85%, more preferably up to about 90% and most preferably up to about 95% of the total color delivered to the hair (Delta E) from a mixture of (a) and (b) on mixing.

17. A process capable of delivering a red shade to hair comprising: application to the hair of a composition according to any of claims 1 to 10 comprising:
(i) from about 0.01% to about 3% by weight of an inorganic peroxygen oxidising agent; and
(ii) from about 0.001% to about 5% by weight of an oxidative hair coloring agent and mixtures thereof;
wherein the untreated hair is prepermed, prebleached light brown hair having L, a, b values of approximately 60, 9 and 32 and wherein the initial shade of the colored hair has a hue value (arc tangent of (b/a)) in the range of from about 25 to about 70, preferably from about 30 to about 65, more preferably from about 35 to about 60 and wherein the initial color intensity (L) is greater than about 10 and less than about 70, preferably greater than about 15 and less than about 65, more preferably greater than about 20 and less than about 60 and wherein the delta E fade is less than about 5.0, preferably less than about 4.5, more preferably less than about 4.0 and wherein the change in hair color, % delta E, after up to 20 washes, is less than about 20%, and preferably less than about 15%, more preferably less than about 10%.

18. A process capable of delivering a brown or black shade to hair comprising: application to the hair of a composition according to any of claims 1 to 10 comprising:
(i) from about 0.01% to about 3% by weight of an inorganic peroxygen oxidising agent; and
(ii) from about 0.001% to about 5% by weight of an oxidative hair coloring agent and mixtures thereof;
wherein the untreated hair is prepermed, prebleached light brown hair having L, a, b values of approximately 60, 9 and 32 and wherein the initial shade of the colored hair has a hue value (arc tangent of (b/a)) of less than about 25, preferably less than about 20 and wherein the initial color intensity (L) is greater than about 1 and less than about 50, preferably greater than about 5 and less than about 45 and wherein the delta E fade is less than about 2.3, preferably less than about 2.0, more preferably less than about 1.7 and wherein the change in hair color, % delta E, after up to 20 washes, is less than about 5%, preferably less than about 4.5%, more preferably less than about 4%, most preferably less than about 3.5%.

19. A process capable of delivering a light brown shade to hair comprising: application to the hair of a composition according to any of claims 1 to 10 comprising:
(i) from about 0.01% to about 3% by weight of an inorganic peroxygen oxidising agent; and
(ii) from about 0.001% to about 5% by weight of an oxidative hair coloring agent and mixtures thereof; and
wherein the untreated hair is prepermed, prebleached light brown hair having L, a, b values of approximately 60, 9 and 32 and wherein the initial shade of the colored hair has a hue value (arc tangent of (b/a)) in the range of from about 70 up to about 110 and wherein the initial color intensity (L) is greater than about 20 and less than about 95, preferably greater than about 25 and less than about 90 and wherein the delta E fade is less than about 2.6, preferably less than about 2.3 and wherein the change in hair color, % delta E, after up to 20 washes, is less than about 15%, preferably less than about 12%, more preferably less than about 10%, most preferably less than about 8%.

20. Use of a composition according to any of claims 1 to 10 for the coloring of textiles and or fibrous substances.
